(19) United States
(12) Patent Application Publication
(10) Pub. No.: US 2007/0092562 A1
(43) Pub. Date: Apr. 26, 2007

(54) Product and process for increasing compactibility of carbohydrates

(75) Inventors: Gary T. Norman, Middletown, DE (US); Cecil W. Propst, Norton Shores, MI (US)

Correspondence Address: MORGAN, LEWIS & BOCKIUS LLP, 1701 MARKET STREET, PHILADELPHIA, PA, 19103-2921, US

(73) Assignee: SPI Pharma, Inc.

(21) Appl. No.: 11/448,656

(22) Filed: Jun. 6, 2006

Related U.S. Application Data
(63) Continuation-in-part of application No. 11/413,674, filed on Apr. 27, 2006.
Application No. 11/413,674 is a continuation of application No. PCT/US04/35982, filed on Oct. 28, 2004.
(60) Provisional application No. 60/515,330, filed on Oct. 28, 2003.

(51) Int. Cl. A61K 31/70; A61K 9/20; A61K 31/045; A61K 31/7012

(52) U.S. Cl. 424/464; 514/23; 514/53; 514/738

(57) ABSTRACT

The present invention includes a method for preparing a highly compactible carbohydrate product, and the product itself. In one embodiment, a composition according to the present invention is a quick-dissolving composition that includes polyols.

### STATEMENT OF RELATED APPLICATIONS

This application is a continuation-in-part of U.S. application Ser. No. 11/413,674, filed on Apr. 27, 2006, which is a continuation of International PCT Application No. PCT/US04/35982, filed on Oct. 28, 2004 (published as WO05/044193, on May 19, 2005), which claims priority to U.S. Provisional Application No. 60/515,330, filed on Oct. 28, 2003, all of which are hereby incorporated by reference in their entirety.

### FIELD OF THE INVENTION

The present invention relates to a process for producing a highly compactible composition including at least two carbohydrates. The present invention also relates to the highly compactible carbohydrate product, and a pharmaceutical composition comprising the product. The present invention also relates to quick-dissolving compositions comprising a highly compactible composition of the present invention.

### BACKGROUND

Carbohydrates are a common ingredient in pharmaceutical compositions as fillers for preparing solid dosage forms, such as tablets. These carbohydrate solid dosage forms are generally prepared using various processes, such as spray-drying, fluid bed granulation, and conventional wet granulation.

### BRIEF SUMMARY OF THE INVENTION

The present invention includes methods for preparing a highly compactible carbohydrate product. The method includes the steps of blending at least a first carbohydrate and a second carbohydrate, wherein the first carbohydrate has a melting point that is higher than the second carbohydrate; melting the second carbohydrate over the first carbohydrate to obtain a highly compacted product; drying the product; and screening the dried product to a desired particle size.

The present invention also includes carbohydrate compositions that include at least a first carbohydrate and a second carbohydrate, wherein the first carbohydrate has a melting point which is greater than said second carbohydrate, the second carbohydrate is uniformly melted over the first carbohydrate.

The present invention includes pharmaceutical compositions that include a carbohydrate composition of the present invention, and at least one of an active ingredient, a lubricant, a flavor, or a color.

The present invention provides a method for producing a carbohydrate composition that is highly compactible and has decreased ejection forces, thereby decreasing the tendency to laminate during tableting.

The present invention includes quick-dissolving pharmaceutical compositions that include a carbohydrate composition of the present invention.

### BRIEF DESCRIPTION OF THE DRAWINGS

FIG. 1 is a graph presenting compactibility of various compositions containing different grades and forms of mannitol, sorbitol, mannitol and sorbitol, and calcium carbonate, all with magnesium stearate as a lubricant, where the black circle indicates one embodiment of a composition of the present invention.

FIG. 2 is a graph representing ejection forces for various grades and forms of mannitol, sorbitol, mannitol and sorbitol, and calcium carbonate, all with magnesium stearate as a lubricant, where the black circle indicates one embodiment of a composition of the present invention.

FIG. 3 is a graph representing compactibility of mannitol (asterisk), mannitol with 3.4 percent sorbitol (black diamond), and mannitol with 5 percent sorbitol (open square), according to the present invention.

FIG. 4 is a schematic representation of an extruder and an extrusion process for use in the present invention.

FIGS. 5A and 5B are schematic representations of a cross-section of tablet. A cross-section of the particle is also depicted (FIG. 5A), including the individual crystals of sorbitol-coated mannitol (FIG. 5B).

FIGS. 6A and 6B are graphs depicting compression force against tablet hardness (FIG. 6A) and tablet hardness against disintegration time (FIG. 6B).

### DETAILED DESCRIPTION

The present invention will be better understood with reference to certain definitions, provided below.

Definitions

As used herein, the term “about” will be understood by persons of ordinary skill in the art and will vary to some extent on the context in which it is used. If there are uses of the term which are not clear to persons of ordinary skill in the art given the context in which it is used, “about” shall mean up to plus or minus 10 percent of the particular value.

As used herein, the term “compactibility” means the loss in volume of a powder being compacted with subsequent gain in mechanical strength of the solid dosage form. As used herein, the terms “solid dosage form,” “tablet,” and “solid preparation” are used synonymously within the context of the present invention. These terms should be construed to include a compacted or compressed composition obtained by compressing or otherwise forming the composition to form a solid having a defined shape.

Detailed Description

The present invention includes a method for preparing a highly compactible carbohydrate product. In one embodiment, the method includes blending at least a first carbohydrate (i.e., a higher melting point carbohydrate) and a second carbohydrate (i.e., a lower melting point carbohydrate), wherein the first carbohydrate has a melting point that is higher than the second carbohydrate, melting the second carbohydrate over the first carbohydrate to create a highly compactible product, drying the product, and screening the product for a desired particle size. Desirable particle sizes range from about 50 microns to about 800 microns, more preferably about 75 microns to about 590 microns, and most preferably, about 100 microns to about 420 microns. In one embodiment of the present invention, the mean particle size is in the range of about 250 microns to about 500 microns.

In one embodiment, the method includes blending at least a first carbohydrate and a second carbohydrate, wherein the first carbohydrate has a melting point that is higher than the second carbohydrate; extruding the carbohydrate blend through an extruder, creating an extrudate; drying the extrudate; and screening the dried extrudate to a desired particle size, wherein the temperature inside said extruder reaches the melting point of the second carbohydrate but not the first carbohydrate.

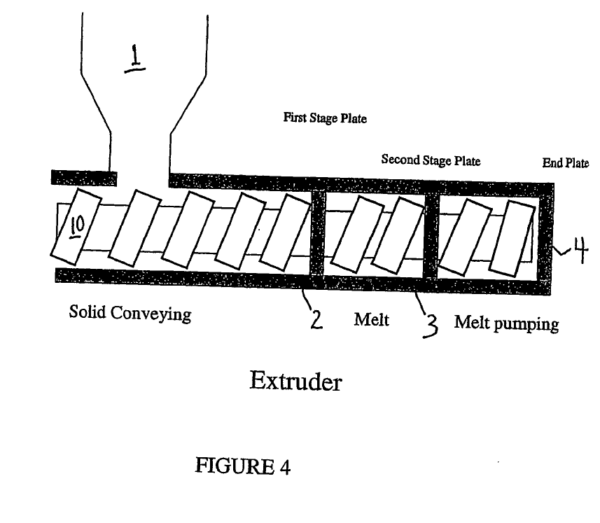

FIG. 4 is a schematic representation of an embodiment of a method of the present invention. The angled rectangles represent screw threads 10. In one embodiment, screw threads 10 have a pitch angle that increases from right to left in FIG. 4. The solid vertical rectangles indicate a first die plate 2, a second die plate 3, and an end die plate 4. In one embodiment, the first and second carbohydrates are fed by one or more feeder lines into a hopper 1, preferably in a rated manner (i.e., the first and second carbohydrates are fed into the hopper 1 in a manner that maintains a specific ratio between the first and second carbohydrates), along with a small amount of water. In one embodiment, blending occurs as the carbohydrates move along the screw path (prior to extrusion through the first die plate 2) as the screw thread pitch of the extruder changes, and/or by pressure mixing caused by mixing of the materials held behind each thread or die plate and the continual flow of additional materials.

In an embodiment of the present invention, the first carbohydrate and the second carbohydrate are different. In another embodiment of the present invention, the first and second carbohydrates may be the same carbohydrate in a different form if the different forms have different melting points. For example, the first carbohydrate may be in crystalline form while the second carbohydrate may be non-crystalline (e.g., amorphous). Alternatively, one or both carbohydrates may be in a spray-dried form or granular form.

The present invention also includes a method for increasing the compactibility of a carbohydrate product. The method includes blending at least a first and a second carbohydrate, the second carbohydrate having a lower melting point temperature than the first carbohydrate, melting the second carbohydrate over the first carbohydrate, drying the product, and screening the product for the desired particle size. Desirable particle sizes range from about 50 microns to about 800 microns, more preferably about 75 microns to about 590 microns, and most preferably, about 100 microns to about 420 microns. In an embodiment of the present invention, the mean particle size is about 250 microns.

In an embodiment according to the present invention, the method includes blending at least a first and a second carbohydrate, each having a different melting point temperature, creating the highly compactible product by extruding the carbohydrate blend through an extruder, drying the extrudate, and screening the extrudate to a desired particle size. Desirable particle sizes range from about 50 microns to about 800 microns, more preferably about 75 microns to about 590 microns, and most preferably, about 100 microns to about 420 microns. In an embodiment of the present invention, the mean particle size is about 250 microns.

In another embodiment of the present invention, the first and second carbohydrate can be continuously fed individually to the extruder and blended during the first stage rather than being blended prior to addition to the extruder.

In another embodiment of the present invention, the first and second carbohydrate are blended prior to addition to hopper 1.

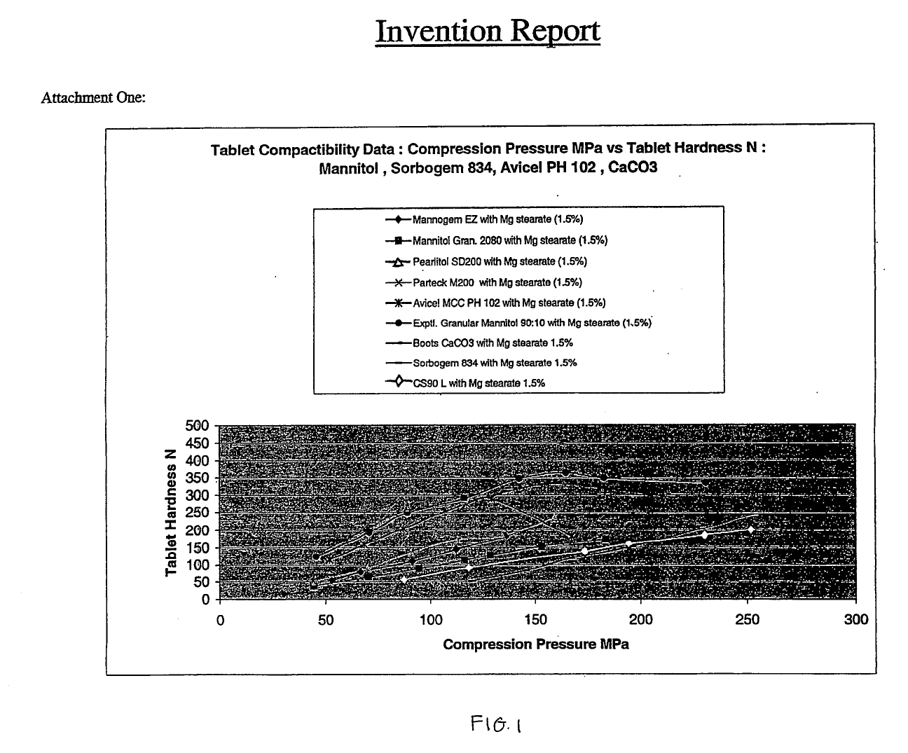

Without being bound to any particular theory, it is believed that the compactibility of a low compactibility carbohydrate can be enhanced by melting a second carbohydrate having a lower melting point over the first carbohydrate. FIG. 1 illustrates that at high compression forces, a composition according to the present invention (indicated by the black circle) maintains high tablet hardness. Changes in the surface characteristics of the first carbohydrate due in part to the underpressure melted flow of the second carbohydrate contribute to the increased compactibility. The second carbohydrate (i.e., the lower melting point carbohydrate) is able to flow over and between the particles of the first carbohydrate (i.e., the higher melting point carbohydrate), creating a more uniformly coated first carbohydrate particle, and a more dense matrix of first carbohydrate particles. In one embodiment, the highly compactible product is useful for preparing, for example, a very robust pharmaceutical tablet because higher compactibility provides greater robustness.

Carbohydrates useful in the present invention include, but are not limited to sugars and polyols, which are sugar alcohols of the general formula CH2OH—(CHOH)n—CH2OH, where n is 2 to 6, and preferably 3 to 6, and their dimeric anhydrides. Preferably, the polyols include, but are not limited to sorbitol, mannitol, erythritol, maltitol, lactitol, isomalt, and mixtures thereof, and sugars such as lactose, xylose, erythrose, fructose, dextrose, sucrose, maltose, and mixtures thereof. In an embodiment of the invention, sugars used in the present invention include xylose, melted over maltose and xylose melted over sucrose. In an embodiment of the invention, polyols used in the present invention include sorbitol melted over mannitol.

In an embodiment according to the present invention, the raw carbohydrate materials are screened through a mesh screen. Mesh screen size can range from about 10 to about 80, preferably from about 20 to about 50, and more preferably about 20 mesh. In one embodiment, after screening, and prior to extrusion, the carbohydrates are mixed uniformly in a V-mixer, preferably 10 cubic feet (Patterson Kelley, East Stroudsburg, Pa.).

In one embodiment according to the present invention, after mixing the carbohydrates and screening them through an appropriately sized mesh screen, the mixture is compacted by extrusion through an extruder, such as the Reitz model RE-6 extruder (Hosokawa Bepex, Minneapolis, Minn.), at an rpm of from about 50 to about 240. The higher the content of the first carbohydrate, the higher the rpm necessary to extrude the product.

In an embodiment of the method of the present invention, the first and second carbohydrates have a minimum difference in melting temperature of from about 20 degrees Celsius to about 80 degrees Celsius, and preferably about 60 degrees Celsius. In one embodiment, the minimum melting temperature is preferably below 120 degrees Celsius, and more preferably below 110 degrees Celsius.

In one embodiment, the carbohydrate composition is extruded through two intermediate die plates (depicted in FIG. 4 as first die plate 2 and second die plate 3), for example, a one-half inch die plate internal to the unit, followed by a one-quarter inch die plate. In one embodiment, the composition is then extruded through an end die plate (depicted in FIG. 4 as end die plate 4), preferably a 12-gauge 0.047-inch 150 hole die plate (17.5 percent openings), as water is continually pumped into the water chamber of the extruder at a rate of about 100 cc/min. The water and the materials are heated to the melting point of the second carbohydrate such that the second carbohydrate melts and forms a solution with the water having a paste-like consistency. Other die plates are also useful in the present invention include any die plate that maintains the extrudate at a temperature of at least within about 10 percent above the melting point of the carbohydrate having the lower melting point.

The water content in the carbohydrate composition typically is equal to or less than about 3 percent, preferably equal to less than 2 percent, and even more equal to preferably less than 1 percent. The product is extruded through the die plate holes in the form of a noodle, wherein the second carbohydrate (i.e., the carbohydrate having a lower melting point) uniformly coats the first carbohydrate (i.e., the carbohydrate having a higher melting point).

The resulting “noodle” is dried in a fluid bed dryer (Fluid Air Model 1000, Aurora, Ill.) to a moisture content of less than 1 percent. The dried carbohydrate composition is screened and milled, for example, on a FITZMILL™ (Fitzpatrick D-6 Mill). In one embodiment, particle size may be from about 2000 to about 50 microns, corresponding to from about 10 to 80 mesh. The dried carbohydrate composition is directly compressible to form a tablet at this point.

In an embodiment of the present invention, the method proceeds as follows:

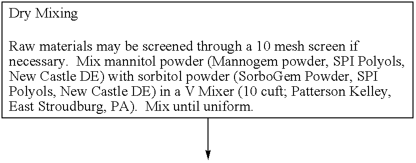

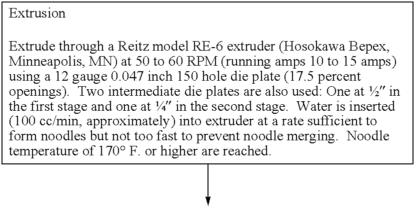

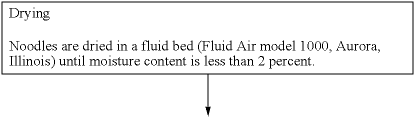

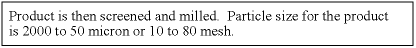

In an embodiment of the invention, the milling process includes use of a cone mill, for example, the Quadro Comil, with a round rod or square impeller, for example. In another embodiment, the mill is a Fitzmill D6 with a knife blade. In one embodiment of the invention, the Fitzmill runs at a speed of from about 2000 rpm to about 5000 rpm. In another embodiment, the Fitzmill runs at a speed of from about 2500 rpm to about 4000 rpm. In another embodiment, the Fitzmill runs at a speed of about 2500 rpm.

In an embodiment of the invention, the milled product is screened using any number of screens, depending on the desired particle size. In one embodiment, the round hole screen is used at one of many different sizes, for example, the 0.079 inch screen or the 0.050 inch screen. In another embodiment, a grader screen is used. In another embodiment, a grader rotor unit for the Fitzmill is used.

In one embodiment of the present invention, the method comprises blending mannitol (melting range between about 164 to 169 degrees C.) and sorbitol (melting range between about 91 and 101 degrees C.) in a 10 cubic foot blender (Patterson Kelley, East Stroudsburg, Pa.). The mannitol product can be from any source, and is preferably MANNOGEM™ powder (SPI Polyols, Inc., New Castle, Del.). Other sources of mannitol powder include GETEC Mannitol powder (BRAZIL), and PEARLITOL™ (Roquette, FRANCE). Preferably, the form of mannitol is a platelike form of crystals, for example, the beta form of mannitol.

The sorbitol product can be from any source, and is preferably SORBOGEM™ powder (SPI Polyols, Inc., New Castle, Del.). Other sources of sorbitol include NeoSorb™ (Roquette, FRANCE), and Sorbitol Instant (Merck & Co., Whitehouse Station, N.J.).

The ratio of mannitol:sorbitol can range from about 97:3 to about 70:30. Preferably, the mannitol to sorbitol ratio is about 80:20, and more preferably, about 90:10, and even more preferably about 97:3.

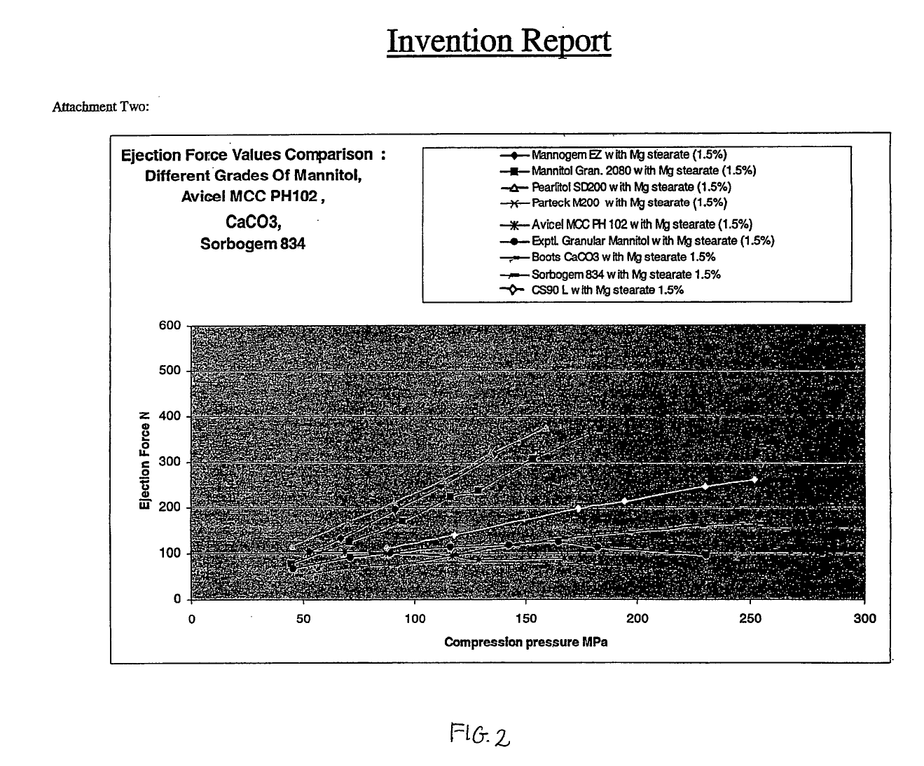

FIGS. 1 and 2 illustrate the superior results obtained with a mannitol/sorbitol composition discussed herein. A composition according to the present invention includes a 90:10 ratio of granular mannitol to crystalline sorbitol and about 1.5 percent magnesium stearate (indicated with a black circle on each graph). Each of the other compositions on the graph in FIGS. 1 and 2 is mannitol, a mannitol/sorbitol combination, or calcium carbonate in various forms are as follows:
(1) spray-dried mannitol with 1.5 percent magnesium stearate commercially available as MANNOGEM EZ (SPI Pharma, New Castle, Del.; solid diamond);
(2) spray-dried mannitol with 1.5 percent magnesium stearate commercially available as PEARLITOL SD200 (Roquette, France; open triangle);
(3) granular mannitol with 1.5 percent magnesium stearate commercially available as Mannitol 2080 (SPI Pharma, New Castle, Del.; square)
(4) spray-dried mannitol with 1.5 percent magnesium stearate and 1.5 percent natural sorbitol impurities, commercially available as Parteck M200 (Merck & Co., Whitehouse Station, N.J.; “X”)
(5) Microcrystalline Cellulose with 1.5 percent magnesium stearate commercially available as AVICEL PH 102 (FMC Corp., Phila., Pa.; asterisk)
(6) boots calcium carbonate with 1.5 percent magnesium stearate (small rectangle)
(7) granular sorbitol commercially available as SORBOGEM 834 (SPI Pharma, New Castle, Del.; large rectangle)
(8) Calcium Carbonate/Starch with 1.5 percent magnesium stearate commercially available as CS90 L (90:10 ratio calcium carbonate:starch, SPI Pharma, New Castle, Del.; open diamond).

The results on each of the graphs indicate that the composition of the present invention has high tablet hardness at relatively low compression forces, and low ejection forces compared with the other products tested. For example, at a compression force of 130 to 140 MPa, a composition of the present invention has sufficient tablet hardness of about 350 newtons (see FIG. 1). Only the Parteck M200 has a higher tablet hardness (about 375 newtons) at the same compression force. All other products tested had lower tablet hardness at the same compression force.

In addition, at the same compression force, a composition of the present invention has the lowest ejection force, at 100 newtons (see FIG. 2), compared with all other products tested. The ejection force for Parteck M200 is around 450 newtons at the same compression force. Therefore, the compositions of the present invention produce a tablet having a high tablet hardness and low ejection force at the same compression pressure, compared with all other products tested. This solves the problem of lamination during tableting.

It is notable that the compositions of the present invention follow similar compactibility and ejection force profiles to that of the SORBOGEM (black bar in FIGS. 1 and 2). This indicates that the surface of the highly compactible composition is sorbitol and the inner core is mannitol. Therefore, the compositions of the present invention enjoy the benefits of the compactibility and ejection force properties of sorbitol without the disadvantages of sorbitol, such as its hygroscopicity, decreased surface area, and increased viscosity, which are all poor characteristics for oral dosage forms. Mannitol/sorbitol compositions according to the present invention are preferable in oral dosage forms of pharmaceutical compositions because (1) these compositions have an increased surface area due to the mannitol, thereby making these compositions dissolve more quickly; (2) mannitol absorbs more calories when it dissolves, thereby producing a cooling effect in the buccal cavity when the compositions dissolve; and (3) viscosity of mannitol is decreased in water, making the mannitol/sorbitol composition diffuse more quickly than a sorbitol composition.

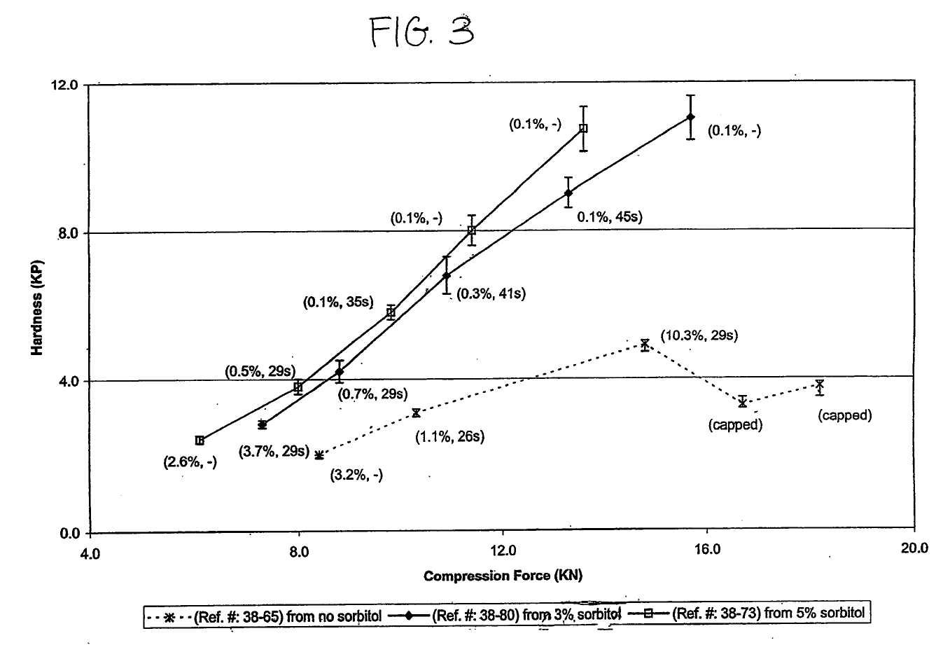

FIG. 3 is a graph representing another data set for compaction and ejection force profiles for compositions according to the present invention. The open boxes indicate a mannitol:sorbitol composition according to the present invention in a ratio of about 95:5. The black diamonds indicate a mannitol:sorbitol composition according to the present invention in a ratio of about 97:3. The asterisks indicate mannitol with less than 1 percent natural impurity sorbitol as a reference point. At each point on the graph, percent friability and disintegration time are noted in parentheses. The data indicate that tablet hardness for the 97:3 mannitol:sorbitol composition steadily increases even after the reference mannitol caps (i.e., the top of the tablet pops off). For example, at about 15 kilonewtons, tablet hardness for the 97:3 composition is about 11 KP, while tablet hardness for the reference mannitol is about 5 KP, and caps at a slightly higher compression force. In addition, the reference mannitol is about 100 times more friable than the 97:3 composition. Therefore, the data in this Figure indicate that a 97:3 mannitol:sorbitol composition has high compactibility (tablet hardness of about 10 KP), yet disintegrates in a short period of time (45 seconds) and has a very low friability (0.1 percent).

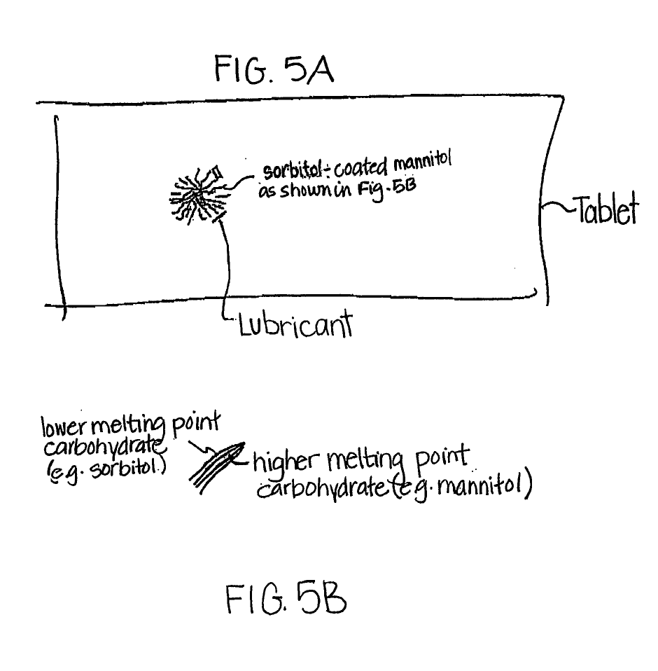

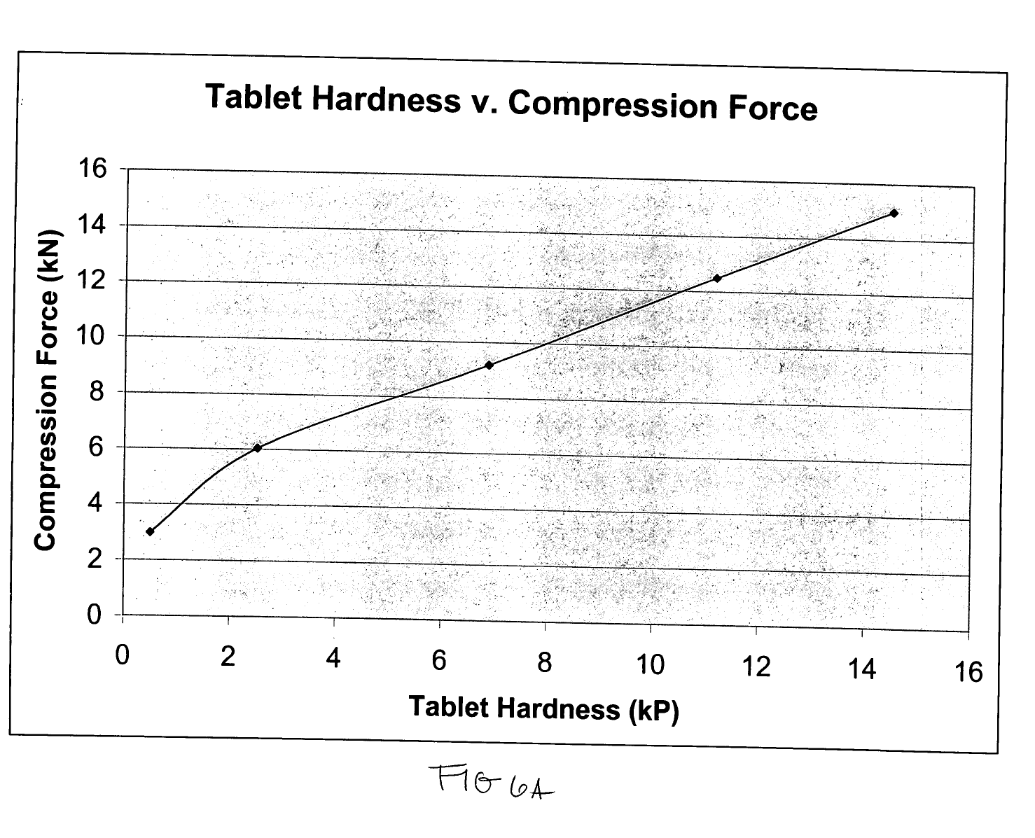

The present invention also includes a highly compactible carbohydrate composition produced by the methods discussed herein. The carbohydrate composition includes a first and a second carbohydrate, wherein the second carbohydrate is melted and coated uniformly over and between the particles of the first carbohydrate. The compositions of the present invention are novel in that they are made up of large particles having a large surface area. By way of example and not by limitation, a 100 micron particle in the composition of the present invention has the surface area characteristic of a 10 micron particle of a single polyol. The increased surface area is due to the fact that these particles are not solid, and internal spaces in the particles exist. A cross-section of a particle of the carbohydrate composition of the present invention, as depicted in FIGS. 5A and 5B, reveals that mannitol is coated with a layer of sorbitol, and the sorbitol-coated mannitol radiates out in the form of spikes from a more dense center. Lubricant is non-uniformly attached to the carbohydrate particle.

The present invention also includes a pharmaceutical composition. The pharmaceutical composition includes a highly compactible carbohydrate composition and one or more of at least one active ingredient, (e.g., calcium carbonate or acetaminophen), a lubricant, a color, and a flavor. The pharmaceutical composition can be in the form of a tablet, a capsule, a liquid, a film, or a gel. Preferably, the pharmaceutical composition is in the form of a tablet.

In one embodiment of the present invention, the pharmaceutical composition dissolves in the buccal cavity in about 60 seconds, preferably within about 45 seconds. In one embodiment, the highly compactible carbohydrate compositions have a mean particle size up to about 250 microns. The mean particle size of the compactible carbohydrate composition can be up to about 800 microns or more or as low as about 50 microns or more. In another embodiment, the compostions have a mean particle size up to about 600 microns. Dissolution time is directly proportional to the mean particle size, such that as the mean particle size of a carbohydrate composition of the present invention increases, the dissolution time also increases.

In an embodiment of the present invention, a quick dissolving orally disintegrating pharmaceutical composition includes a carbohydrate composition of the present invention in an amount of from about 1 percent to about 99 percent of the total pharmaceutical composition. In another embodiment, the carbohydrate composition of the present invention is present in an amount of from about 10 percent to about 90 percent. In another embodiment, the carbohydrate composition of the present invention is present in an amount of from about 20 percent to about 80 percent. In another embodiment, the carbohydrate composition of the present invention is present in an amount of from about 30 percent to about 70 percent. In another embodiment, the carbohydrate composition is present in an amount of from about 50 percent to about 80 percent. In another embodiment, the carbohydrate composition of the present invention is present in an amount of from about 40 percent to about 60 percent. In another embodiment, the carbohydrate composition of the present invention is present in an amount of about 50 percent.

In another embodiment, a quick dissolving orally disintegrating pharmaceutical composition includes a disintegrant. Any disintegrant known in the art is useful in the present invention, including povidone, crospovidone, carmellose, sodium croscarmellose, sodium starch glycolate, and combinations thereof. Examples of commercially available disintegrants include POLYPLASDONE XL™ (crospovidone; ISP Technologies, Wayne, N.J.), EXPLOTAB™ (sodium starch glycolate; JRS Pharma, Patterson, N.Y.), and AC-DI-SOL™ (sodium croscarmellose; FMC Corporation, Philadelphia, Pa.).

In an embodiment of the present invention, a disintegrant is present in a quick dissolving orally disintegrating pharmaceutical composition in an amount of from about 0 percent to about 50 percent of the total pharmaceutical composition. In another embodiment, a disintegrant is present in an amount of from about 5 percent to about 40 percent. In another embodiment, a disintegrant is present in an amount of from about 10 percent to about 30 percent. In another embodiment, a disintegrant is present in an amount of from about 10 percent to about 20 percent.

In another embodiment, a quick dissolving orally disintegrating pharmaceutical composition includes a cellulose. Any cellulose known in the art is useful in the present invention, including microcrystalline cellulose, amorphous cellulose, and combinations thereof. Examples of commercially available microcrystalline celluloses include AVICEL™ PH102 (microcrystalline cellulose; FMC Corporation, Philadelphia, Pa.) and VIVAPUR™ (microcrystalline cellulose; JRS Pharma, Patterson, N.Y.). Amorphous celluloses are readily available from a number of different manufacturers.

In an embodiment of the present invention, a cellulose is present in a quick dissolving orally disintegrating pharmaceutical composition in an amount of from about 0 percent to about 50 percent of the total pharmaceutical composition. In another embodiment, a cellulose is present in an amount of from about 5 percent to about 40 percent. In another embodiment, a cellulose is present in an amount of from about 10 percent to about 30 percent. In another embodiment, a cellulose is present in an amount of from about 10 percent to about 20 percent.

In another embodiment, a quick dissolving orally disintegrating pharmaceutical composition includes a lubricant. Any lubricant known in the art is useful in the present invention, including magnesium stearate, sodium stearyl fumarate, glyceryl behenate and combinations thereof. Examples of commercially available lubricants include LUBRIPHARM™ (sodium stearyl fumarate; SPI Pharma, New Castle, Del.) and PRUV® (sodium stearyl fumarate; JRS Pharma, Patterson, N.Y.).

In an embodiment of the present invention, a lubricant is present in a quick dissolving orally disintegrating pharmaceutical composition in an amount of from about 10 percent to about 0 percent of the total pharmaceutical composition. In another embodiment, a lubricant is present in an amount of from about 1 percent to about 5 percent. In another embodiment, a lubricant is present in an amount of from about 1 percent to about 3 percent. In another embodiment, a lubricant is present in an amount of about 2 percent.

In another embodiment, a quick dissolving orally disintegrating pharmaceutical composition includes a glidant. Any glidant known in the art is useful in the present invention, including silica gel, colloidal silica, colloidal silica dioxides, precipitated silica, and combinations thereof. Examples of commercially available glidants include SIPERNAT™ S50 (silicon dioxide; DeGussa, France), CAB-O-SIL™ (fumed silica; Cabot Corporation, Boston, Mass.), and Aerosil™ (fumed silica; DeGussa, France).

In an embodiment of the present invention, a glidant is present in a quick dissolving orally disintegrating pharmaceutical composition in an amount of from about 0 percent to about 10 percent of the total pharmaceutical composition. In another embodiment, a glidant is present in an amount of from about 0.5 percent to about 5 percent. In another embodiment, a glidant is present in an amount of from about 0.5 percent to about 3 percent. In another embodiment, a glidant is present in an amount of about 1 percent.

In one embodiment of the present invention, a sweetener is included in a quick dissolving orally disintegrating pharmaceutical composition in an amount of from about 0 percent to about 10 percent of the total pharmaceutical composition. In an embodiment of the present invention, a sweetener is present in a range of from about 0.01 percent to about 1 percent. In another embodiment, a sweetener is present in an amount of from about 0.05 percent to about 0.5 percent. In another embodiment, a sweetener is present in an amount of about 0.3 percent. Sweeteners useful in the present invention include, but are not limited to natural sweeteners, high intensity sweeteners, and artificial sweeteners, including but not limited to sucralose, aspartame, fructose, dextrose, dextrin, maltodextrin, corn syrup, high fructose corn syrup, saccharin, sucrose, acesulsame potassium, and glucose.

In another embodiment of the present invention, a color or flavor is present in a quick-dissolving orally disintegrating pharmaceutical composition. There is no limitation on color or flavor that is useful in the present invention, and these characteristics will likely be chosen based on the age of the patient consuming the pharmaceutical composition. Those of skill in the art will know which colors and flavors are useful in the present invention and the percent range of each present in the composition of the present invention. Color and flavor are inert ingredients and generally do not have any effect on the efficacy of the pharmaceutical composition.

In another embodiment of the present invention, an active ingredient is included in a quick-dissolving orally disintegrating pharmaceutical composition. Any active ingredient is useful in a quick-dissolving composition according to the present invention. Examples of active ingredients are described herein. The amount of active present in the pharmaceutical composition of the present invention will depend, in part, on the type of active ingredient. In an embodiment of the invention, a coated active is used. In another embodiment, an uncoated active is used. In an embodiment of the present invention, the amount of active ingredient included in the quick-dissolving orally disintegrating pharmaceutical composition is 0.01 percent to 80 percent of the total weight of the pharmaceutical composition.

In an embodiment of the present invention, a quick dissolving orally disintegrating pharmaceutical composition comprises:

| Ingredient | Percent | mg/Tablet |
|---|---|---|
| Carbohydrate Composition | 54.30 | 271.50 |
| Cellulose | 20.00 | 100.00 |
| Disintegrant | 20.00 | 100.00 |
| Flavor | 2.00 | 10.00 |
| Sweetener | 0.50 | 2.50 |
| Lubricant | 2.20 | 11.00 |
| Glidant | 1.00 | 5.00 |
| TOTAL | 100.0 | 500.0 |

In an embodiment of the present invention, the pharmaceutical composition includes the carbohydrate composition of the present invention in a range of from about 30 percent to about 99 percent by weight of the pharmaceutical composition.

Due to the high compactibility of a carbohydrate composition according to the present invention, the pharmaceutical composition typically requires less lubricant than a conventional pharmaceutical composition. The lubricant may be present in a pharmaceutical composition according to the present invention at about 0.1 percent to about 2 percent of the total pharmaceutical composition. Preferably, the lubricant is present at less than about 1 percent. Lubricants useful in the present invention include, but are not limited to sodium stearyl fumarate, glyceryl behenate, and magnesium stearate (“flow aids”). Lubricant attaches non-uniformly to the carbohydrate particles that make up the carbohydrate composition of the present invention.

In one embodiment, calcium carbonate is included as an active ingredient in a composition according to the present invention. Calcium carbonate is present in a range of from about 5 percent to about 40 percent, preferably from about 10 percent to about 30 percent, and more preferably about 20 percent.

In one embodiment, a sweetener may also be included in the composition of the present invention, and is preferably added to chewable tablets. Sweeteners may be present in a range of from about 0.01 percent to about 1 percent, preferably from about 0.05 percent to about 0.5 percent, and more preferably about 0.3 percent. Sweeteners useful in the present invention include, but are not limited to sucralose, aspartame, fructose, dextrose, dextrin, maltodextrin, corn syrup, high fructose corn syrup, saccharin, sucrose, acesulsame potassium, and glucose.

There is no limitation on color or flavor that is useful in the present invention, and these characteristics will likely be chosen based on the age of the patient consuming the pharmaceutical composition. Those of skill in the art will know which colors and flavors are usefill in the present invention and the percent range of each present in the composition of the present invention. Color and flavor are inert ingredients and generally do not have any effect on the efficacy of the pharmaceutical composition.

In one embodiment of the composition according to the present invention, acetaminophen (APAP) is included as an active ingredient in a pharmaceutical composition according to the present invention. APAP is present in a range of from about 1 percent to about 30 percent, preferably from about 7 percent to about 25 percent, and more preferably about 14 percent.

In one embodiment of the present invention, a pharmaceutical composition according to the present invention includes:
Carbohydrate composition 62 percent
Calcium carbonate 20 percent
92 percent APAP 14.5 percent
Flavor 2 percent
Sweetener 0.3 percent
Color 0.3 percent
Lubricant 1 percent

Active ingredients useful in the composition of the present invention also include, but are not limited to pharmaceutical ingredients and nutraceutical ingredients. Examples of pharmaceutical ingredients that can be used include, but are not limited to gastrointestinal function conditioning agents, including, but not limited to bromopride, metoclopramide, cisapride, and domperidone; anti-inflammatory agents, including, but not limited to aceclofenac, diclofenac, flubiprofen, sulindac, and celecoxib; analgesics, including, but not limited to acetaminophen and aspirin; agents for erectile dysfunction therapy, including, but not limited to sildenafil and apomorphine; anti-migraines, including, but not limited to sumatriptan and ergotamin; antihistaminic agents, including, but not limited to loratadine, fexofenadine, pseudoephedrine and cetirizine; cardiovascular agents, including, but not limited to nitroglycerine and isosorbide dinitrate; diuretics, including, but not limited to furocemide and spironolactone; anti-hypertensive agents, including, but not limited to propranolol, amlodipine, felodipine, nifedipine, captoprile, ramiprile, atenolol, and diltiazem; anti-hypolipidemic agents, including, but not limited to simvistatin, atrovastatin, and pravastatin; anti-ulcer agents, including, but not limited to cimietidine, ranitidine, famotidine, omeprazole, and lansoprazol; anti-emetics, including, but not limited to meclizine hydrochoride, ondansetron, granisetron, ramosetron, and tropisetron; anti-asthmatic agents, including, but not limited to aminophylline, theophylline, terbuttaline, fenoterol, formoterol, and ketotifen; anti-depressants, including, but not limited to fluoxetine and sertraline; vitamins, including, but not limited to B1, B2, B6, B12 and C; anti-thrombotic agents, including, but not limited to sulfinpyrazone, dipyridamole, and ticlopidine; chemotherapeutic agents, including, but not limited to cefaclor, bacampicillin, sulfamethoxazole, and rifampicin; hormones, including, but not limited to dexamethasone and methyltestosterone; anti-helminthic agents, including, but not limited to piperazine, ivermectine, and mebendazole; and anti-diabetic agents, including, but not limited to acarbose, gliclazid, and glipizid.

Preferable pharmaceutical ingredients which may be used in the present invention include, but are non limited to acetaminophen, pseudoephedrine hydrochloride, dextromethorphan hydrobromide, dompereidone, famotidine, meclizine hydrochloride, scopolamine hydrobromide, ondansetron, cisapride, granisetron, sildenafil, loratadine, and amlodipine.

Examples of nutraceutical ingredients include, but are not limited to any ingredient that is thought to have a beneficial effect on human health. Such ingredients include coenzyme Q-10, chondroitoin, echinacea, ephedra, glucosamine, garlic, ginkgo biloba, ginseng, grape seed extract, guarana, hawthorn, herbs, kava, kola nut, lutein, St. John's wort, vinpocetine, and yohimbe.

The following example is given to illustrate the present invention. It should be understood, however, that the invention is not to be limited to the specific conditions or details described in this example. Throughout the specification, any and all references to a publicly available document, including but not limited to a U.S. patent, are specifically incorporated by reference.

### EXAMPLE 1

Quick-dissolving Composition

The carbohydrate composition (trademarked by SPI Pharma, Inc. as COMPRESSOL S™) was prepared using the method set forth above. Briefly, mannitol powder (Mannogem powder, SPI Polyols, New Castle Del.) and sorbitol powder (SorboGem Powder, SPI Polyols, New Castle Del.) were mixed uniformly in a V Mixer (10 cubic foot; Patterson Kelley, East Stroudburg, Pa.). The mixture was then extruded through a Reitz model RE-6 extruder (Hosokawa Bepex, Minneapolis, Minn.) at 50 to 60 RPM (running amps 10 to 15 amps) using a 12 gauge 0.047 inch 150 hole die plate (17.5 percent openings). Two intermediate die plates were also used: one at a half-inch in the first stage and one at a quarter-inch in the second stage. Water was inserted at approximately 100 cubic centimeters per minute into the extruder at a rate sufficient to form noodles. The extruded noodles were at a temperature of about 170 degrees Fahrenheit or higher. Noodles were dried in a fluid bed (Fluid Air model 1000, Aurora, Ill.) until the moisture content was less than 2 percent. The noodles were then milled using a Fitzmill D6 and sieved through a 20 mesh screen to achieve the desired particle size.

The pharmaceutical composition (SPI Pharma, Inc. Ref. No.45-164) was then prepared by first sieving the Sipernat™ 50S (DeGussa, France) through a 30 mesh screen. The Compressol™ S and the Sipernat™ 50S were blended for 1 minute using a Turbula blender. The Avicel PH02, crospovidone XL, SD Peppermint Flavor, sucralose, and lubricant (LUBRIPHARM™; SPI Pharma Inc., New Castle, Del.) were then sieved through a 30 mesh sieve (up to about 600 microns), combined with the Compressol/Sipemat mixture, and blended for 5 minutes using the Turbula blender. The composition was then pressed into tablets using a rotary press fitted with a single punch and die set of round faced tools at 0.4375 inches. Five groups of samples were produced, each at a different compression force: 3 KN, 6 KN, 9 KN, 12 KN, and 15 KN. The table below indicates the amounts of each ingredient in the tablets in each sample group:

| INGREDIENT | Percent | mg/Tablet |
|---|---|---|
| Compressol S (Carbohydrate Composition) | 54.30 | 271.50 |
| Avicel PH102 | 20.00 | 100.00 |
| Crospovidone XL | 20.00 | 100.00 |
| SD Peppermint Flavor 3100627 (A. M. Todd) | 2.00 | 10.00 |
| Sucralose | 0.50 | 2.50 |
| Lubripharm | 2.20 | 11.00 |
| Sipernat 50S | 1.00 | 5.00 |
| TOTAL | 100.00 | 500.0 |

Friability, disintegration time, hardness, and compactibility were measured for each variation of the compression force.

Tablet hardness was determined using a Dr. Schleuniger Pharmatron Model 6D (Dr. Schleuniger Pharmatron, Switzerland) hardness tester. Five tablets were individually tested and results were recorded. Standard deviation and average hardness were calculated.

Disintegration time was determined using the Erweka ZT71 tester (Erweka GmbH, Heusenstamm, Germany) equipped with baskets, disks, and a vessel containing water at 100 degrees Fahrenheit. Three tablets were tested and average disintegration time was reorded.

Tablet Friability was determined using an Erweka TA10 tester (Erweka GmbH, Heusenstamm, Germany) using 100 rotations. the weight of 10 tablets was measured before and after being dropped by the friabilator. The percent friability was calculated and recorded.

Tablet thickness was determined using a Mitutoyo micrometer (Mitutoyo USA, New Castle, Del.). Five tablets were individually tested and results were recorded. The average and standard deviations were calculated.

The results of these experiments follow.

### Sample Group 1

The target compression force for sample group 1 was 3 KN. The actual compression force achieved was 2.978 KN. The ejection force for this tablet was 107.6 N, and the press speed was 20.7 RPM. The table below demonstrates the hardness, friability, and disintegration time for five samples. As noted, the friability test for tablets compressed at 3 KN is not sufficient for a quick dissolving tablet.

| | | | Hardness | Friability | Disintegration |
|---|---|---|---|---|---|
| Sample No. | Weight (mg) | Thickness (mm) | (KP) | (%) | Time (sec) |
| 1-1 | 510 | 6.08 | 0.5 | Fails | 15 |
| 1-2 | 506 | 6.03 | 0.5 | | |
| 1-3 | 510 | 6.06 | 0.5 | | |
| 1-4 | 510 | 6.05 | 0.5 | | |
| 1-5 | 505 | 6.1 | 0.5 | | |
| Mean | 508.2 | 6.06 | 0.5 | | |
| Standard | 2.49 | 0.03 | 0.00 | | |
| Deviation | | | | | |
| RSD | 0.49 | 0.45 | 0.00 | | |

### Sample 2

The target compression force for sample group 2 was 6 KN. The actual compression force achieved was 6.036 KN. The ejection force for this tablet was 153.8 N, and the press speed was 20.7 RPM. The table below demonstrates the hardness, friability, and disintegration time for five samples.

| | | Thickness | Hardness | Friability | ODT |
|---|---|---|---|---|---|
| Sample No. | Weight (mg) | (mm) | (KP) | (%) | (sec) |
| 2-1 | 513 | 5.35 | 2.6 | 1.60 | 42 |
| 2-2 | 511 | 5.34 | 2.7 | | |
| 2-3 | 514 | 5.34 | 2.5 | | |
| 2-4 | 512 | 5.33 | 2.5 | | |
| 2-5 | 514 | 5.33 | 2.3 | | |
| Mean | 512.8 | 5.34 | 2.52 | | |
| Standard | 1.30 | 0.01 | 0.15 | | |
| Deviation | | | | | |
| RSD | 0.25 | 0.16 | 5.89 | | |

### Sample 3

The target compression force for sample group 3 was 9 KN. The actual compression force achieved was 9.19 KN. The ejection force for this tablet was 186.4 N, and the press speed was 20.7 RPM. The table below demonstrates the hardness, friability, and disintegration time for five samples.

| | | | | | Disinte- |
|---|---|---|---|---|---|
| Sample | | Thickness | Hardness | Friability | gration |
| No. | Weight (mg) | (mm) | (KP) | (%) | Time (sec) |
| 3-1 | 516 | 4.89 | 7.4 | 0.00 | 110 |
| 3-2 | 511 | 4.86 | 7 | | |
| 3-3 | 514 | 4.88 | 6.8 | | |
| 3-4 | 514 | 4.87 | 6.5 | | |
| 3-5 | 519 | 4.85 | 6.7 | | |
| Mean | 514.8 | 4.87 | 6.88 | | |
| Standard | 2.95 | 0.02 | 0.34 | | |
| Deviation | | | | | |
| RSD | 0.57 | 0.32 | 4.97 | | |

### Sample 4

The target compression force for sample group 4 was 12 KN. The actual compression force achieved was 12.468 KN. The ejection force for this tablet was 206.6 N, and the press speed was 20.7 RPM. The table below demonstrates the hardness, friability, and disintegration time for five samples.

| | | | | | Disinte- |
|---|---|---|---|---|---|
| Sample | | Thickness | Hardness | Friability | gration |
| No. | Weight (mg) | (mm) | (KP) | (%) | Time (sec) |
| 4-1 | 507 | 4.515 | 11.8 | 0.00 | 175 |
| 4-2 | 504 | 4.554 | 10.3 | | |
| 4-3 | 506 | 4.535 | 11.2 | | |
| 4-4 | 506 | 4.524 | 11.6 | | |
| 4-5 | 505 | 4.519 | 11.0 | | |
| Mean | 505.6 | 4.529 | 11.2 | | |
| Standard | 1.14 | 0.02 | 0.58 | | |
| Deviation | | | | | |
| RSD | 0.23 | 0.35 | 5.23 | | |

### Sample Group 5

The target compression force for sample group 5 was 15 SKN. The actual compression force achieved was 15.004 KN. The ejection force for this tablet was 213.6 N, and the press speed was 20.7 RPM. The table below demonstrates the hardness, friability, and disintegration time for five samples.

| | | | | | Disinte- |
|---|---|---|---|---|---|
| Sample | | Thickness | Hardness | Friability | gration |
| No. | Weight (mg) | (mm) | (KP) | (%) | Time (sec) |
| 5-1 | 508 | 4.31 | 15.2 | 0.00 | >300 |
| 5-2 | 504 | 4.40 | 13.8 | | |
| 5-3 | 506 | 4.40 | 14.8 | | |
| 5-4 | 506 | 4.40 | 14.6 | | |
| 5-5 | 505 | 4.40 | 14.1 | | |
| Mean | 505.8 | 4.38 | 14.5 | | |
| Standard | 1.48 | 0.04 | 0.56 | | |
| Deviation | | | | | |
| RSD | 0.29 | 0.87 | 3.84 | | |

The data can be summarized as follows:

| Compression | | Disintegration | |
|---|---|---|---|
| Force (KN) | Tablet Hardness (KP) | Time (Sec) | Friability (%) |
| 2.978 | 0.5 | 15 | Fail |
| 6.036 | 2.52 | 42 | 1.6 |
| 9.19 | 6.88 | 110 | 0 |
| 12.5 | 11.18 | 175 | 0 |
| 15.004 | 14.5 | 300 | 0 |

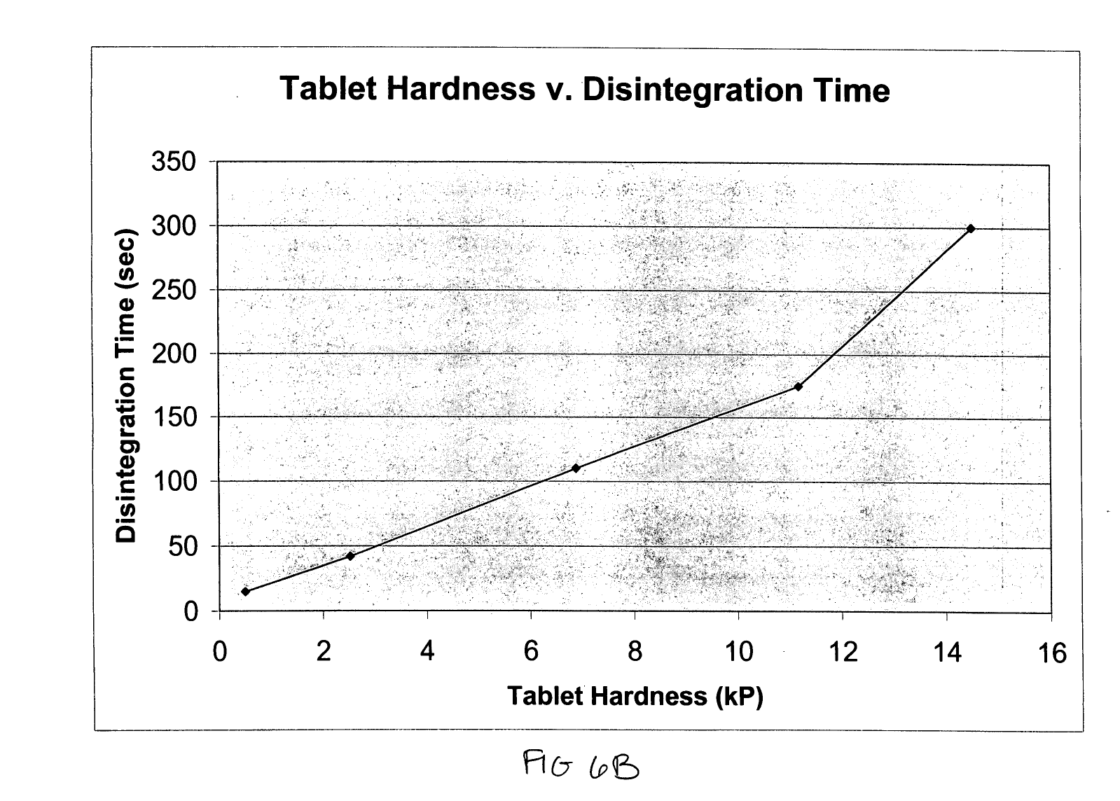

The summary data is depicted in graph form in FIGS. 6A and 6B. The summary data demonstrate that as compression force increases, the tablet hardness increases, and friability decreases. As tablet hardness increases, disintegration time also increases. In an embodiment of the present invention, an optimal quick-dissolving tablet would have a high tablet hardness, low friability, and a disintegration time of less than about 2 minutes. As shown in FIGS. 6A and 6B, several optimal quick-dissolving formulations fall within the range of a tablet hardness of about 2.52 and about 6.88, friability of about 1.6 percent or less, and a disintegration time of less than about 110 seconds.

It should be understood that the invention is not to be limited to the specific conditions or details described herein. Throughout the specification, any and all references to a publicly available document, including but not limited to a U.S. patent, are specifically incorporated by reference.

It will be apparent to those skilled in the art that various modifications and variations can be made in the methods and compositions of the present invention without departing from the spirit or scope of the invention. Thus, it is intended that the present invention cover the modifications and variations of the present invention provided they come within the scope of the appended claims and their equivalents.

What is claimed is:

1. A highly compactible, quick-dissolving carbohydrate composition, the composition comprising a disintegrant and at least a first carbohydrate and a second carbohydrate, wherein said first carbohydrate has a melting point which is greater than said second carbohydrate, and said second carbohydrate is melted over said first carbohydrate.

2. The composition of claim 1, wherein said first and second carbohydrates are selected from the group consisting of mannitol, sorbitol, maltitol, xylitol, lactitol, isomalt, erythritol, maltose, sucrose, fructose, lactose, and xylose.

3. The composition of claim 1, wherein said first carbohydrate is mannitol and said second carbohydrate is sorbitol.

4. The composition of claim 3, wherein said mannitol is present in a range of from about 70 percent to about 97 percent, and wherein said sorbitol is present in a range of from about 3 percent to about 30 percent.

5. The composition of claim 4 having a water content of about 1 percent.

6. The composition according to claim 1, further comprising a lubricant.

7. The composition of claim 6, wherein said lubricant is selected from the group consisting of sodium stearyl fumarate and magnesium stearate.

8. The composition of claim 6, wherein said lubricant is magnesium stearate.

9. The composition of claim 1, further comprising a glidant.

10. The composition of claim 9, wherein said glidant is silica.

11. The composition of claim 1, further comprising a cellulose.

12. The composition of claim 1, wherein said disintegrant is crospovidone.

13. The composition of claim 1, further comprising an active ingredient.
